(19) United States
(12) Patent Application Publication
(10) Pub. No.: US 2010/0234605 A1
(43) Pub. Date: Sep. 16, 2010

(54) METHODS AND COMPOSITIONS FOR PRODUCING DIFLUOROMETHYLENE-AND TRIFLUOROMETHYL-CONTAINING COMPOUNDS

(75) Inventors: Teruo Umemoto, Denver, CO (US); Rajendra P. Singh, Denver, CO (US)

Correspondence Address: SWANSON & BRATSCHUN, L.L.C., 8210 SOUTHPARK TERRACE, LITTLETON, CO, 80120, US

(73) Assignee: IM&T RESEARCH, INC., Denver, CO (US)

(21) Appl. No.: 12/305,868

(22) PCT Filed: Dec. 9, 2008
(86) PCT No.: PCT/US08/86044

§ 371 Date: Dec. 19, 2008

Related U.S. Application Data
(60) Provisional application No. 61/007,076, filed on Dec. 11, 2007.
(60) Provisional application No. 61/007,097, filed on Dec. 11, 2007.

(51) Int. Cl. C07D 213/74; C07C 17/361; C07C 41/22; C07C 319/20; C07C 17/18

(52) U.S. Cl. 546/304; 570/140; 568/663; 568/683; 568/665; 568/56; 570/142

(57) ABSTRACT

New methods for producing difluoromethylene-containing compounds with phenylsulfur trifluoride or a primary alkyl-substituted phenylsulfur trifluoride are disclosed. Also, new methods for producing trifluoromethyl-containing compounds with phenylsulfur trifluoride or primary alkyl-substituted phenylsulfur trifluoride are also disclosed.

### TECHNICAL FIELD

The present invention relates to difluoromethylene- and trifluoromethyl-containing compounds and to the compositions and methods for producing the same.

### BACKGROUND OF THE INVENTION

Fluorine-containing compounds have found wide use in medical, agricultural, electronic and other like industries. Difluoromethylene (CF₂)— and trifluoromethyl (CF₃)— containing compounds are particularly useful in these industries as each type of compound shows specific biologic activity or physical properties based on the unique electronic and steric effects of the CF₂ and CF₃ fluorine atoms [see, for example, Chemical & Engineering News, June 5, pp. 15-32 (2006); J. Fluorine Chem., Vol. 127 (2006), pp. 992-1012; Tetrahedron, Vol. 52 (1996), pp. 8619-8683; Angew. Chem. Ind. Ed., Vol. 39, pp. 4216-4235 (2000)]. However, although highly useful, CF₂ and CF₃ containing compounds are not typically natural to the environment, requiring such compounds to be prepared through organic synthesis. This has proven to be a major obstacle to the use of the CF₂ and CF₃ containing compounds, as each type of compound has proven difficult and expensive to synthesis.

Difluoromethylene-containing compounds are typically prepared using methodologies as described in Tetrahedron, Vol. 52 (1996), pp. 8619-8683. The most general and useful methodology for preparation of CF₂-containing compounds has been conversion of a carbonyl group (C═O) or its derivative groups or moieties (e.g., thiocarbonyl group (C═S), dithioketal or dithioacetal (S—C—S)), to a difluoromethylene group (CF₂). There are an enormous number of known compounds having a carbonyl group; their derivation to thiocarbonyl, dithioketal, and/or dithioacetal compounds has also proven feasible. However, as discussed in more detail below, use of this conventional methodology has significant drawbacks based on safety, cost, yield, reactivity, selectivity, number of reactants, applicability, and/or difficulty of application to commercial production. The present invention provides significant and unexpected improvements over these conventional methodologies, as is discussed in more detail below. Similar concerns exist for conventional preparation of CF₃-containing compounds, including drawbacks based on safety, cost, yield, reactivity, selectivity, number of reactants, applicability, and/or difficulty of application for commercial production.

In more detail, CF₂-containing compounds have been conventionally prepared by conversion of a carbonyl group, thiocarbonyl group, dithioketal moiety, or a dithioacetal moiety to a difluoromethylene group. These methods and their drawbacks include: (1) reaction of a carbonyl-containing compound with sulfur tetrafluoride (SF₄), however, SF₄ is a highly toxic gas (bp −40° C.) that must be utilized under pressure for the reaction to proceed [J. Am. Chem. Soc., Vol. 82, pp. 543-551 (1960)]; (2) reaction of a carbonyl-containing compound with phenylsulfur trifluoride, however, reaction of ketones and aliphatic aldehydes provides low yields, and hence provides only limited usefulness for this reaction [J. Am. Chem. Soc., Vol. 84, pp. 3058-3063 (1962)]; (3) reaction of a carbonyl- or thiocarbonyl-containing compound with diethylaminosulfur trifluoride (DAST), however, DAST is an unstable liquid having a highly explosive nature [J. Org. Chem., Vol. 40, pp. 574-57 (1975); J. Org. Chem., Vol. 55, pp. 768-770 (1990); Chem. & Eng. News, Vol. 57, No. 19, p. 4 (1979)]; (4) reaction of a carbonyl- or thiocarbonyl-containing compound with bis(2-methoxyethyl)aminosulfur trifluoride (Deoxo-Fluor®) or its N-aryl analogs, however, Deoxo-Fluor® and the N-aryl analogs are compounds having low thermal stability [see the following discussion and Table 1, and U.S. Pat. No. 6,222,064 B1; Chem. Commun., Vol. 1999, pp. 215-216; J. Org. Chem. Vol. 65, pp. 4830-4832 (2000)]; (5) reaction of a carbonyl-containing compound with selenium tetrafluoride (SeF₄), however, use of selenium compounds tend to be highly toxic and unsafe [J. Am. Chem. Soc., Vol. 96, pp. 925-927 (1974)], or with various other designed fluorinating agents that provide greater safety but have provided substantially reduced reactivity and yields; e.g., α,α-difluoroalkylamino reagents [CF₂HCF₂NMe₂, J. Fluorine Chem., Vol. 109, pp. 25-31 (2001); 2,2-difluoro-1,3-dimethylimidazolidine, Chem. Commun., Vol. 2002, pp. 1618-1619; and N,N-diethyl-α,α-difluoro-(m-methylbenzyl)amine, J. Fluorine Chem., Vol. 126, pp. 721-725 (2005)]; (6) reaction of a thiocarbonyl-containing compound or a dithioketal, a halogenating agent such as 1,3-dibromo-5,5-dimethylhydantoin (DBH), N-bromosuccinimide (NBS) or N-iodosuccinimide (NIS), and a fluoride source such as a mixture of hydrogen fluoride and pyridine [pyridine poly(hydrogen fluoride)] or tetrabutylammonium dihydrogentrifluoride [(C₄H₉)₄NH₂F₃], however, this method requires three reactants and has a drawback that side reactions, such as bromination of a substrate, can be prevalent [J. Org. Chem., Vol. 51, pp. 3508-3513 (1986); Synlett, Vol. 1994, pp. 251-252; Tetrahedron Lett., Vol. 35, pp. 3983-3984 (1994); Synlett, Vol. 1991, pp. 909-910; Chem. Lett., pp. 827-830 (1992); Tetrahedron Lett., Vol. 33, pp. 4173-4176 (1992)]; (7) reaction of a dithioketal, 1-(chloromethyl)-4-fluoro-1,4-diazoniabicyclo[2.2.2]octane bis(tetrafluoroborate), and pyridine poly(hydrogen fluoride), however, this method requires three reactants including expensive reagents, and has a further drawback that side reactions such as hydrolysis can be prevalent in the reaction, and this method cannot be applied to dithioacetals because of the occurrence of exclusive hydrolysis [Chem. Commun., Vol. 2005, pp. 654-656]; (8) reaction of a dithioketal with p-iodotoluenedifluoride, however, p-iodotoluenedifluoride is expensive and separation of a difluoromethylene product from p-iodotoluene (from p-iodotoluenedifluoride) is difficult due to products being collected in the organic layer in the extraction process [Synlett, Vol. 1991, pp. 191-192]; (9) reaction of a dithioketal, sulfuryl chloride, and pyridine poly(hydrogen fluoride), however, this method requires three reactants and a fluoride source, pyridine poly(hydrogen fluoride), which is needed in a large excess, and the process has a crucial drawback that side reaction such as chlorination can be prevalent [Synlett, Vol. 1993, pp. 691-693]; (10) reaction of a thiocarbonyl-containing compound or a dithioacetal with BrF₃, however, BrF₃ is a strong oxidizer which must be treated with great care and has to be prepared from molecular fluorine (F₂), a hard to handle, dangerous compound [Chem. Commun, Vol. 1993, pp. 1761-1762; Org. Lett., Vol. 5 (2003), pp. 769-771]; (11) reaction of a dithioketal with N-iodosuccinimide or 1,3-dibromo-5,5-dimethylhydantoin, hexafluoropropene-diethylamine reagent, and water, but this method requires four reactants, and a fluoride source, hexafluoropropene-diethylamine reagent, which is expensive [J. Fluorine Chem., Vol. 71, pp. 9-12 (1995)]; (12) reaction of a dithioketal with a F₂-iodine mixture, however, this method requires F₂ a dangerous compound to utilize [J. Chem. Soc., Perkin Trans. 1, Vol. 1994, pp. 1941-1944]; and finally (13) electrolysis of a dithioketal or dithioacetal in the presence of triethylamine trihydrofluoride, however, applicability of this method is narrow due to low selectivity and yield (as a result of the electrolysis reaction) [Chem. Left, Vol. 1992, p. 1995].

With regard to preparation of CF₃-containing compounds, several conventional processes have been utilized, including: (1) reaction of a carboxylic acid or a fluoroformate with sulfur tetrafluoride (SF₄), as noted previously, SF₄ is a highly toxic gas (bp −40° C.) when utilized under pressure [J. Am. Chem. Soc., Vol. 82, pp. 543-551 (1960)]; (2) reaction of a chlorothioformate with tungsten hexafluoride (WF₆), however, WF₆ is expensive, highly toxic and exists in a state of being almost a gas at room temperatures (boiling point (bp) 17° C.) [Tetrahedron Letters, pp. 2253-2256 (1973)]; (3) reaction of a carboxylic acid with diethylaminosulfur trifluoride (DAST), DAST is an unstable liquid having a highly explosive nature [see, for example, U.S. Pat. No. 3,914,265 and Chem. & Eng. News, Vol. 57, No. 19, p 4 (1979)]; (4) reaction of a carbonyl fluoride or dithiocarbamate with bis(2-methoxyethyl)aminosulfur trifluoride (Deoxo-Fluor®), Deoxo-Fluor® has low thermal stability [see the following discussion and Table 1, and Chemical Communications, pp. 215-216 (1999); J. Org. Chem. Vol. 65, pp. 4830-4832 (2000)]; (5) reaction of a dithiocarboxylate, xanthate, or dithiocarbamate with 1,3-dibromo-5,5-dimethylhydantoin (DBH) or N-bromosuccinimide (NBS) or N-iodosuccinimide (NIS) and tetrabutylammonium fluoride-hydrogen fluoride [α]₄N⁺F⁻ (HF)₂] or a mixture of hydrogen fluoride and pyridine [pyridine poly(hydrogen fluoride)] [Chemistry Letters, pp. 827-830 (1992); Tetrahedron Letters, Vol. 33, pp. 4173-4176 and 4177-4178 (1992)], this method includes side reactions such as a bromination of the substrate, resulting in reduced yields, or requires expensive reagents such as NIS; (6) reaction of a trichloromethyl-substituted compound with metal fluorides such as SbF₃/SbF₂Cl₂ [see, for example, J. Am. Chem. Soc., Vol. 73, pp. 1042-1043 (1951)], the starting materials are limited and the application is limited because of extremely acidic reaction conditions; (7) reaction of a trichloromethyl-substituted compound with hydrogen fluoride (HF) [see, for example, J. Am. Chem. Soc., Vol. 60, p. 492 (1938) and Vol. 76, 2343-2345 (1954)], the starting materials are limited and it is problematic that a large amount of gaseous and toxic hydrogen chloride (HCl) is evolved from the reaction mixture, including HF which is highly toxic and exists in a state of being almost a gas (bp 19.5° C.); (8) reaction of phenol or its derivative with carbon tetrachloride and HF [J. Org. Chem., Vol. 44, pp. 2907-2910 (1979)], however, yields are poor and large amounts of HCl are evolved from HF, again a troublesome development; (9) reaction of an organic compound with a nucleophilic, radical, or electrophilic trifluoromethylating agent, which is expensive and availability limited, in addition, the selectivity of reaction is low and the substrates usable are limited [Journal of Fluorine Chemistry, Vol. 128, pp. 975-996 (2007)]; and (10) reaction of benzene derivatives possessing an electron-donating substituent with carbon tetrachloride and HF [J. Org. Chem., Vol. 44, 2907 (1979)], in addition to the problem of evolving a large amount of HCl, this reaction gives an isomeric mixture of hard to separate reaction products.

In addition, other CF₃-containing compound production methods include: (11) reaction of an alkanecarboxylic acid with phenylsulfur trifluoride giving a low yield of a (trifluoromethyl)alkane [J. Am. Chem. Soc., Vol. 84, pp. 3058-3063 (1962)]; and finally, and more recently; (12) a reaction of a carboxylic acid and a reactive multi-alkylated phenylsulfur trifluoride as reported in U.S. Pat. No. 7,265,247 B1, incorporated by reference herein for all purposes.

Each of the above discussed CF₂ and CF₃-containing compound production methods has room for improvement on providing a safe, simple, effective, selective, and widely applicable method. As such, there is a need in the field to provide safe, reactive, selective, simple, less hazardous, cost effective, widely applicable methods for producing high yields using easily available starting materials.

The present invention is directed toward overcoming one or more of the problems discussed above.

### SUMMARY OF THE INVENTION

The present invention provides new methods for production of difluoromethylene-containing compounds from sulfur-containing compounds, e.g., thiocarbonyl-containing compounds, dithioketals, and dithioacetals, which are themselves easily available or prepared from carbonyl-containing compounds. The difluoromethylene-containing compounds have been shown to have tremendous potential in medical, agricultural, electronic and other like uses. Novel difluoromethylene-containing compounds are also provided.

The present invention also provided methods for the production of trifluoromethyl-containing compounds from substrates which are readily available or prepared. The trifluoromethyl-containing compounds have been shown to have tremendous potential in medical, agricultural, and electronic uses, as well as in other like materials and/or uses. Novel trifluoromethyl-containing compounds are also provided.

These and various other features and advantages of the invention will be apparent from a reading of the following detailed description and a review of the appended claims.

### DETAILED DESCRIPTION OF THE INVENTION

### Difluoromethylene-Containing Compounds

The present invention provides novel methods for producing difluoromethylene-containing compounds, represented by the formula R¹CF₂R², from a sulfur-containing compound, represented by the formula R¹—C(R³)(R⁴)—R². The difluoromethylene-containing compounds are useful in medical, agricultural, biological, electronic and other like fields. Unlike previous production methods in the art, the present invention is safe, simple, low cost and produces high yields of target difluoromethylene-containing compounds.

In one embodiment, a method for preparing a difluoromethylene-containing compound represented by R¹CF₂R², comprises reacting a sulfur-containing compound, represented by R¹—C(R³)(R⁴)—R², with an arylsulfur trifluoride, represented by ArSF₃.

This reaction is described by the following scheme:

R¹—C(R³)(R⁴)—R²+ArSF₃→R¹CF₂R²

For purposes of R¹CF₂R² and R¹—C(R³)(R⁴)—R², R¹ is an organic moiety and R² is a hydrogen atom or an organic moiety. Organic moieties of R¹ and R² may be different or the same. R³ and R⁴ each can be independently an alkylthio group, an arylthio group, or an aralkylthio group, or R³ and R⁴ can combine to form a sulfur atom. When R³ and R⁴ each is independently an alkylthio group, an arylthio group, or an aralkylthio group, R³ and R⁴ may be combined or connected via an alkylene chain and/or a hetero atom(s).

Ar is phenyl group or phenyl group having a primary alkyl substituent, wherein the primary alkyl substituent has one to eight carbon atoms.

In addition, when R³ and R⁴ combine to form S (a sulfur atom), the compounds represented by R¹—C(R³)(R⁴)—R² may be described by a formula: R¹—C(═S)—R².

For purposes of the present invention, an organic moiety of R¹ or R² is composed of a carbon atom(s) and a hydrogen atom(s) with or without an oxygen atom(s), a nitrogen atom(s), a sulfur atoms(s), a phosphorous atom(s), and/or another hetero atom(s); R¹ and R² are selected to not hinder the reaction(s) of the invention. Preferable examples of the organic moiety of R¹ or R² include: substituted or unsubstituted alkyl, alkyloxy, alkylthio, alkylamino, and dialkylamino groups; substituted or unsubstituted aryl, aryloxy, arylthio, arylamino, diarylamino, and aryl(alkyl)amino groups; substituted or unsubstituted heteroaryl, heteroaryloxy, heteroarylthio, heteroarylamino, di(heteroaryl)amino, heteroaryl(alkyl)amino, and heteroaryl(aryl)amino groups; substituted or unsubstituted alkenyl groups; substituted or unsubstituted alkynyl groups; and other like group(s).

The term “alkyl” as used herein refers to linear, branched, or cyclic alkyl groups. The term “substituted alkyl” as used herein refers to an alkyl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

The term “substituted aryl” as used herein refers to an aryl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

The term “substituted heteroaryl” as used herein refers to a heteroaryl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

The term “substituted alkeny” as used herein refers to an alkenyl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

The term “substituted alkynyl” as used herein refers to an alkynyl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

Substituted alkyl's as used in “substituted alkyloxy,” “substituted alkylthio,” “substituted alkylamino,” “substituted dialkylamino,” “substituted aryl(alkyl)amino,” and “substituted heteroaryl(alkyl)” are the same as or equivalent to “substituted alkyl” as described above. Similarly, substituted aryl's as used in “substituted aryloxy,” “substituted arylthio,” “substituted arylamino,” “substituted diarylamino,” “substituted aryl(alkyl)amino,” and “substituted heteroaryl(aryl)amino,” are the same as or equivalent to “substituted aryl” described above. Similarly, substituted heteroaryl's as used in “substituted heteroaryloxy,” “substituted heteroarylthio,” “substituted heteroarylamino,” “substituted di(heteroaryl)amino,” “substituted heteroaryl(alkyl)amino,” and “substituted heteroaryl(aryl)amino,” are the same as or equivalent to “substituted heteroaryl” as described above.

R¹ and R² groups of R′—C(R³)(R⁴)—R² as starting materials may be different from R¹ and R² of R¹CF₂R² as products, respectively. Thus, this invention can include transformation of a R¹ group to a different R¹ group or of a R² group to a different R² group. Transformation can take place under the reaction conditions herein or during the reaction of the present invention together with transformation of the —C(R³)(R⁴)— group to a CF₂ group by the arylsulfur trifluoride represented by ArSF₃.

Preferable examples of alkylthio groups of R³ and R⁴ include: methylthio, ethylthio, n-propylthio, iso-propylthio, n-butylthio, sec-butylthio, iso-butylthio, tert-butylthio, and so on. Methylthio, ethylthio, and n-propylthio are more preferable because of relative availability. Preferable examples of arylthio groups of R³ and R⁴ include phenyl thio, o-, m-, and p-tolylthio, o-, m-, and p-chlorophenylthio, o-, m-, and p-bromophenylthio, and so on. Phenylthio is more preferable due to its relative low cost. Preferable examples of aralkylthio groups of R³ and R⁴ include benzylthio, o-, m-, and p-methylbenzylthio, o-, m-, and p-chlorobenzylthio, o-, m-, and p-bromobenzylthio, 1-phenylethylthio, 2-phenylethylthio, and so on. Benzylthio is more preferable due to its relative low cost.

When R³ and R⁴ are combined or connected via an alkylene chain and/or a hetero atom(s), preferable examples of R₃ and R₄ include the following; —SCH₂CH₂S—, —SCH₂CH₂CH₂S—, —SCH(CH₃)CH₂S—, —SCH₂CH₂CH₂CH₂S—, —SCH₂CH(CH₃)CH₂S—, —SCH(CH₃)CH₂CH₂S—, —SCH₂CH₂OCH₂CH₂S—, and so on, and —SCH₂CH₂S— and —SCH₂CH₂CH₂S— are more preferable due to relative availability.

R¹—C(R³)(R⁴)—R² as used herein is commercially available or can be prepared from carbonyl-containing compounds or other compounds according to conventional methods [see, for example, Synthesis, Vol. 1973, pp. 149-151; Tetrahedron, Vol. 41, pp. 5061-5087 (1985); Methoden Der Organishen Chemie (Houben-weyl), Vierte Auflage; Georg Thieme Verlag Stattgart, New York (1985), Band E5 (Teil 2) pp. 891-916; J. Org. Chem., Vol. 51, pp. 3508-3513 (1986); Synthetic Communications, Vol. 19, pp. 547-552 (1989); Organic Letters, Vol. 5, pp. 767-771 (2003), each of which is incorporated by reference in their entirety for all purposes].

As described herein, Ar of ArSF₃ is a phenyl group or a phenyl group having a primary alkyl substituent having one to eight carbons, preferably, one to four carbons. Preferable examples of ArSF₃ include: phenylsulfur trifluoride, o, m, and p-methylphenylsulfur trifluoride (or o, m, and p-tolylsulfur trifluoride), o, m, and p-ethylphenylsulfur trifluoride, o, m, and p-(n-propyl)phenylsulfur trifluoride, o, m, and p-(n-butyl)phenylsulfur trifluoride, o, m, and p-(2-methylpropyl)phenylsulfur trifluoride, o, m, and p-(n-pentyl)phenylsulfur trifluoride, o, m, and p-(n-hexyl)phenylsulfur trifluoride, o, m, and p-(n-heptyl)phenylsulfur trifluoride, and o, m, p-(n-octyl)phenylsulfur trifluoride. Among them, phenylsulfur trifluoride, p-methylphenylsulfur trifluoride, p-ethylphenylsulfur trifluoride, p-(n-propyl)phenylsulfur trifluoride, p-(n-butyl)phenylsulfur trifluoride, and p-(2-methylpropyl)phenylsulfur trifluoride are more preferable, and phenylsulfur trifluoride (PhSF₃) and p-methylphenylsulfur trifluoride (p-CH₃C₆H₄SF₃) are furthermore preferred, and phenylsulfur trifluoride is most preferred because of its relative low cost.

ArSF₃ used herein can be prepared with ease at high yield, and with low cost according to the methods described in the literature [see, for example, Synthetic Communications, Vol. 33, pp. 2505-2509 (2003), which is incorporated herein by reference in its entirety for all purposes].

Reactions as described herein can be conducted with or without a solvent. In some embodiments, the reaction can proceed mildly and selectively with a solvent. Solvents are preferably exemplified as hydrocarbons such as hexane, cyclohexane, heptane, octane, nonane, decane, and so on; halocarbons such as methylene chloride, chloroform, carbon tetrachloride, dichloroethane, trichloroethane, tetrachloroethane, perfluorohexane, perfluoroheptane, perfluorooctane, perfluorononane, perfluoro(methylcyclohexane), perfluoro-1-methyldecaline, perfluoro-2-butyltetrahydrofuran, Fluorinart® FC-40˜FC-104, and so on; ethers such as diethyl ether, dipropyl ether, diisopropyl ether, dibutyl ether, diisobutyl ether, di(sec-butyl)ether, tert-butyl methyl ether, tetrahydrofuran, dioxane, dimethoxyethane, diglyme, triglyme, and so on; aromatices such as benzene, toluene, chlorobenzene, dichlorobenzene, hexafluorobenzene, benzotrifluoride, bis(trifluoromethyl)benzene, and so on; esters such as ethyl acetate, methyl acetate, methyl propionate, and so on; or a mixture or combination of two or more solvents mentioned above. Mixture combination of solvents can be at any ratio as long as they function for their intended use.

In some embodiments, yield is optimized by addition of about one mole or more of ArSF₃ per mole of R¹—C(R³)(R⁴)—R². The amount of ArSF₃ can be chosen in the range of from about 1 to about 5 moles of ArSF₃ and more preferably from about 1 to about 3 moles of ArSF₃, especially where cost is a concern.

In order to optimize product yield reaction temperatures are performed in the range of from about −50° C. to about +150° C. More typically, the reaction temperature is from about −30° C. to about +120° C., and furthermore, preferably from about −10° C. to about +100° C.

Reaction time varies dependent upon reaction temperature, and the types and amounts of substrate, reagent, and solvent. As such, reaction time is generally determined as the amount of time required to complete a particular reaction, but can be from about 0.1 hours to about several days.

Embodiments of the invention can be conducted in an open or substantially sealed (closed) reactor, and are preferably conducted under dry conditions as ArSF₃ is consumed by reaction with moisture or water.

In other embodiments, reactions of the invention can be conducted in the presence of hydrogen fluoride or a mixture of hydrogen fluoride and an amine compound(s), which may accelerate the reaction. The hydrogen fluoride may be in situ generated by addition of a necessary amount of water or an alcohol such as methanol, ethanol, propanol, butanol, and so on. The water or alcohol is added into the reaction mixture, since ArSF₃ reacts with water or an alcohol to generate hydrogen fluoride, as shown in the following reaction equations, however, this in situ generation method of hydrogen fluoride requires ArSF₃ be consumed at equimolar amounts of water or alcohol.

ArSF₃+H₂O→2HF+ArSOF

or

ArSF₃+CₙH₂ₙ₊₁OH(n=1˜4)→HF+CₙH₂ₙ₊₁F(n=1˜4)+ArSOF.

The mixture of hydrogen fluoride and amine compound(s) is preferably exemplified by a mixture of hydrogen fluoride and pyridine (for example, a mixture of about 70 wt % HF and about 30 wt % pyridine) or a mixture of hydrogen fluoride and triethylamine [for example, a 3:1 (molar ratio) mixture of hydrogen fluoride and triethylamine, Et₃N(HF)₃]. The amount of hydrogen fluoride or a mixture of hydrogen fluoride and an amine compound(s) may be a catalytic amount to an excess amount for the reaction of this invention, dependent on reaction conditions.

The reactions of the invention may also be conducted in the presence of a tetraalkylammonium fluoride-hydrogen fluoride such as tetrabutylammonium fluoride-hydrogen fluoride [for example, tetrabutylammonium dihydrogentrifluoride, (C₄H₉)₄NH₂F₃]. The amount of a tetraalkylammonium fluoride-hydrogen fluoride may be a catalytic amount to an excess amount for the reaction of this invention, dependent on reaction conditions.

In some cases, in order to restrain decomposition of starting material(s) and/or products sensitive to acidic conditions, the reaction(s) of the invention may be conducted in the presence of a base such as metal fluorides, e.g., lithium fluoride, sodium fluoride, potassium fluoride, cesium fluoride, and so on, and amines such as pyridine, methylpyridine, dimethylpyridine, trimethylpyridine, chloropyridine, triethylamine, and so on.

Methods of the invention are safe and simple, and easily applicable to industrial production. Industrial herein refers to an amount necessary for large scale use or sale as compared to research amounts. A variety of sulfur-containing compounds, represented by R¹—C(R³)(R⁴)—R², as starting materials are easily available or prepared. The arylsulfur trifluorides used in the present invention can be prepared in high yields from inexpensive diphenyl disulfide or primary alkyl-substituted diphenyl disulfides with less expensive reagents, e.g., potassium fluoride and chlorine gas, according to the known methods mentioned above. In addition, as shown below, the arylsulfur trifluorides show very high thermal stability compared to conventional SF₃ reagents such as diethylaminosulfur trifluoride (Et₂NSF₃; DAST) and bis(2-methoxyethyl)aminosulfur trifluoride [(CH₃OCH₂CH₂)₂NSF₃; Deoxy-Fluor®] (which have been used for the preparation of the difluoromethylene-containing compounds, see Background above).

Table 1 shows thermal analysis data for PhSF₃ and p-CH₃C₆H₄SF₃ used in the present invention, together with conventional compounds: DAST and Deoxo-Fluor® (included for comparison). Decomposition temperature and exothermic heat (−ΔH) of each compound was determined using Differential Scanning Spectroscopy, i.e., using a Differential Scanning Spectrometer (DSC). The decomposition temperature is the temperature at which onset of decomposition begins, and the exothermic heat is the amount of heat that results from the compounds decomposition. In general, a higher decomposition temperature and lower exothermic heat value is indicative of a compound having greater thermal stability and safety.

Table 1 illustrates that compounds used in embodiments of the present invention, phenylsulfur trifluoride and p-methylphenylsulfur trifluoride, show very high decomposition temperature and low exothermic heat values over the conventional fluorinating agents, DAST and Deoxo-Fluor®. This data illustrates that the present invention's methods are greatly improved for safety over other conventional methods, e.g., DAST and Deoxo-Fluor®. This is a significant and unexpected improvement over prior art production procedures.

TABLE 1
Thermal Analysis Data of Phenylsulfur Trifluoride (PhSF₃),
p-CH₃C₆H₄SF₃, DAST, and Deoxo-Fluor ®

| | Decomposition | |
|---|---|---|
| Compound | temp. (° C.) | −ΔH(J/g) |
| PhSF₃ | 305 | 826 |
| p-CH₃C₆H₄SF₃ | 274 | 1096 |
| (C₂H₅)₂NSF₃ (DAST) | ~140 | 1700 |
| (CH₃OCH₂CH₂)₂NSF₃ (Deoxo-Fluor ®) | ~140 | 1100 |

As provided by the present invention, difluoromethylene-containing compounds can be safely, easily and cost-effectively produced from available starting materials.

### Trifluoromethyl-Containing Compounds:

Embodiments of the present invention also provide new methods for producing trifluoromethyl-containing compounds, represented by RCF₃, from a carbon-containing compound., represented by R—C(=A)-R^{a}. Trifluoromethyl-containing compounds are useful in medical, agricultural, biological, and electronic material uses, as well as in other like field. Unlike previous methods in the art, embodiments of the present invention are unexpectedly safe, easy, and low cost for preparation of highly selective and enhanced yields of trifluoromethyl-containing compounds.

In one embodiment, a method of preparing a trifluoromethyl-containing compound, RCF₃, comprises reacting a carbon-containing compound, represented by R—C(=A)-R^{a}, with an arylsulfur trifluoride, represented by ArSF₃:

R—C(=A)-R^{a}+ArSF₃→RCF₃

For purposes herein and directed toward the trifluoromethyl-containing compounds: R is an organic moiety; A is a sulfur atom; R^{a} is SR^{b}, wherein R^{b} is a hydrogen atom, an alkyl group, an aryl group, an aralkyl group, a silyl group, a metal atom, an ammonium moiety, a phosphonium moiety, or S—C(═S)—R wherein R is the same as above.

With regard to ArSF₃ for use in producing trifluoromethyl-containing compounds, Ar is a phenyl group or a phenyl group having a primary alkyl substituent, wherein the primary alkyl substituent has from one to eight carbon atoms.

When R—C(=A)-R^{a} is a thiocarbonyl-containing compound represented by the formula R—C(═S)—SR^{b}, then the reaction scheme is described as follows:

R—C(═S)—SR^{b}+ArSF₃→RCF₃

With respect to trifluoromethyl-containing compounds and the schemes above, R is an organic moiety composed of a carbon atom(s) and a hydrogen atom(s) with or without oxygen atom(s), nitrogen atom(s), sulfur atom(s), phosphorous atom(s), and/or other hetero atom(s). R is selected to not hinder (or have limited hindrance) on the reaction(s) of the invention. Preferable examples of the organic moiety of R include: substituted or unsubstituted alkyl, alkyloxy, alkylthio, alkylamino, and dialkylamino groups; substituted or unsubstituted aryl, aryloxy, arylthio, arylamino, diarylamino, and aryl(alkyl)amino groups; substituted or unsubstituted heteroaryl, heteroaryloxy, heteroarylthio, heteroarylamino, di(heteroaryl)amino, heteroaryl(alkyl)amino, and heteroaryl(aryl)amino groups; substituted or unsubstituted alkenyl groups; substituted or unsubstituted alkynyl groups; and other like groups.

The term “alkyl” as used herein refers to a linear, branched, or cyclic alkyl. The term “substituted alkyl” as used herein refers to an alkyl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, again which do not substantially limit reactions of this invention.

The term “substituted aryl” as used herein refers to an aryl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

The term “substituted heteroaryl” as used herein refers to a heteroaryl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

The term “substituted alkeny” as used herein refers to an alkenyl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

The term “substituted alkynyl” as used herein refers to an alkynyl moiety having one or more substituents such as a halogen atom, a substituted or unsubstituted alkyl group, a substituted or unsubstituted aryl group, a substituted or unsubstituted heteroaryl group, a substituted or unsubstituted alkenyl group, a substituted or unsubstituted alkynyl group, and/or an O, N, S, P, and/or any other one or more heteroatoms-containing group, which do not substantially limit reactions of this invention.

Substituted alkyl's as used in “substituted alkyloxy,” “substituted alkylthio,” “substituted alkylamino,” “substituted dialkylamino,” “substituted aryl(alkyl)amino,” and “substituted heteroaryl(alkyl)” are the same as or equivalent to “substituted alkyl” described above. Similarly, substituted aryl's appearing in “substituted aryloxy,” “substituted arylthio,” “substituted arylamino,” “substituted diarylamino,” “substituted aryl(alkyl)amino,” and “substituted heteroaryl(aryl)amino,” are the same as or equivalent to “substituted aryl” as described above. Similarly, substituted heteroaryl's appearing in “substituted heteroaryloxy,” “substituted heteroarylthio,” “substituted heteroarylamino,” “substituted di(heteroaryl)amino,” “substituted heteroaryl(alkyl)amino,” and “substituted heteroaryl(aryl)amino,” are the same as or equivalent to “substituted heteroaryl” described above.

The R group in R—C(═S)—SR^{b} may be different from the R group of RCF₃ in any given reaction as products. Thus, embodiments of this invention include transformation of R to another R, which may take place under reaction conditions herein or during the reaction of the present invention, as long as the C(═S)—SR^{b} group is transformed to a CF₃ group by the arylsulfur trifluoride represented by ArSF₃.

Preferable examples of alkyl groups of R^{b} include methyl, ethyl, n-propyl, iso-propyl, n-butyl, sec-butyl, iso-butyl, tert-butyl and so on. Methyl, ethyl, and propyl are more preferable because of availability. Preferable examples of aryl groups of R^{b} include: phenyl, o, m, and p-tolyl, o, m, and p-chlorophenyl, o, m, and p-bromophenyl, and so on. Phenyl is more preferable due to relative cost. Preferable examples of aralkyl groups of R^{b} include: benzyl, o, m, and p-methylbenzyl, o, m, and p-chlorobenzyl, o, m, and p-bromobenzyl, 1-phenylethyl, 2-phenylethyl, and so on. Benzyl is preferable because of relative low cost. Preferable examples of silyl groups of R² include alkyl, aralkyl, and/or aryl-substituted silyl groups such as trimethylsilyl, triethylsilyl, tri(n-propyl)silyl, tri(n-butyl)silyl, t-butyldimethylsilyl, di(isopropyl)methylsilyl, benzyl(dimethyl)silyl, triphenylsilyl, dimethylphenylsilyl, and so on. Trimethylsilyl and triethylsilyl are more preferable due to relative availability.

Preferable examples of metal atoms of R^{b} include alkali metals, alkali earth metals, transition metals and so on. Alkali metals such as Li, Na, and K and transition metals such as ½Zn and ½Cu are preferable. Preferable examples of ammonium moieties of R^{b} include ammonium (NH₄), methylammonium, ethylammonium, propylammonium, butylammonium, diethylammonium, trimethylammonium, triethylammonium, tripropylammonium, tributylammonium, pyrrolidinium, piperidinium, tetramethylammonium, tetraethylammonium, tetrapropylammonium, tetrabutylammonium, benzyltrimethylammonium, benzyltriethylammonium, and so on. Ammonium, diethylammonium, triethylammonium, tetramethylammonium, tetraethylammonium, and benzyltrimethylammonium are more preferable due to relative availability. Preferable examples of phosphonium moieties of R^{b} include tetramethylphosphonium, tetraethylphosphonium, tetrapropylphosphonium, tetrabutylphosphonium, tetraphenylphosphonium, and so on. Tetraphenylphosphonium is more preferable due to relative availability.

Ar of ArSF₃ is a phenyl group or a phenyl group having a primary alkyl substituent having one to eight carbons, preferably, one to four carbons. Preferable examples of ArSF₃ include: phenylsulfur trifluoride, o, m, and p-methylphenylsulfur trifluoride (or o, m, and p-tolylsulfur trifluoride), o, m, and p-ethylphenylsulfur trifluoride, o, m, and p-(n-propyl)phenylsulfur trifluoride, o, m, and p-(n-butyl)phenylsulfur trifluoride, o, m, and p-(2-methylpropyl)phenylsulfur trifluoride, o, m, and p-(n-pentyl)phenylsulfur trifluoride, o, m, and p-(n-hexyl)phenylsulfur trifluoride, o, m, and p-(n-heptyl)phenylsulfur trifluoride, and o, m, p-(n-octyl)phenylsulfur trifluoride. Among them, phenylsulfur trifluoride, p-methylphenylsulfur trifluoride, p-ethylphenylsulfur trifluoride, p-(n-propyl)phenylsulfur trifluoride, p-(n-butyl)phenylsulfur trifluoride, and p-(2-methylpropyl)phenylsulfur trifluoride are more preferable, and phenylsulfur trifluoride (PhSF₃) and p-methylphenylsulfur trifluoride (p-CH₃C₆H₄SF₃) are furthermore preferred, and phenylsulfur trifluoride is most preferred because of its relative low cost.

The ArSF₃ used in this invention can be prepared at high yield, and low cost, according to methods provided in the literature [see, for example, Synthetic Communications, Vol. 33, No. 14, pp. 2505-2509 (2003), which is incorporated by reference herein in its entirety].

Thiocarbonyl-containing compounds, represented by R—C(═S)—SR^{b}, as starting materials are easily available or prepared according to conventional methods [see, for example, Synthesis, Vol. 1973, pp. 149-151; Tetrahedron, Vol. 41, pp. 5061-5087 (1985); Methoden Der Organishen Chemie (Houben-weyl), Vierte Auflage; Georg Thieme Verlag Stattgart, New York (1985), Band E5 (Teil 2) pp. 891-916; Synthetic Communications, Vol. 19, pp. 547-552 (1989)] each of which is incorporated by reference in its entirety herein.

In order to obtain good product yields, the reaction temperature is typically in the range of from about −50° C. to about +150° C. More typically, the reaction temperature is from about −30° C. to about +120° C., and furthermore, about −10° C. to about +100° C.

In order to obtain optimal product yield, ArSF₃ is used in an amount of about 2 moles or more per mole of thiocarbonyl-containing compound as represented by R—C(—S)—SR^{b}. Preferably, about 2 to about 8 moles of ArSF₃ can be used, and more preferably about 2 to about 5.5 moles can be used, especially where cost is a concern.

The reaction time for trifluoromethyl-containing compounds is dependent upon reaction temperature, and the types and amounts of substrate, reagent, and solvent. As such, reaction time is generally determined as the amount of time required to complete a particular reaction, but can be from about 0.1 hours to about several days.

In one embodiment herein, a reaction of the invention is conducted in the presence of hydrogen fluoride or a mixture of hydrogen fluoride and an amine compound(s), (used to accelerate the reaction). The hydrogen fluoride may be in situ generated by adding a necessary amount of water or an alcohol such as methanol, ethanol, propanol, butanol, and so on, into the reaction mixture. ArSF₃ reacts with water or an alcohol to generate hydrogen fluoride as shown in the following reaction equations, however, this in situ generation method of hydrogen fluoride requires an amount of ArSF₃ that is equimolar to water or an alcohol be consumed.

ArSF₃+H₂O→2HF+ArSOF

or

ArSF₃+CₙH₂ₙ₊₁+OH(n=1˜4)→HF+CₙH₂ₙ₊₁F(n=1˜4)+ArSOF

The mixture of hydrogen fluoride and amine compound(s) is preferably exemplified by a mixture of hydrogen fluoride and pyridine (for example, a mixture of about 70 wt % HF and about 30 wt % pyridine) or a mixture of hydrogen fluoride and triethylamine [for example, a 3:1 (molar ratio) mixture of hydrogen fluoride and triethylamine, Et₃N(HF)₃]. The amount of hydrogen fluoride, or a mixture of hydrogen fluoride and an amine compound(s), may be from a catalytic amount to an excess amount.

In some cases, in order to restrain decomposition of starting material(s) and/or products sensitive to acidic conditions, the reaction of the invention may be conducted in the presence of a base such as metal fluorides, e.g., lithium fluoride, sodium fluoride, potassium fluoride, cesium fluoride, and so on, and/or amines such as pyridine, methylpyridine, dimethylpyridine, trimethylpyridine, chloropyridine, triethylamine, and so on. The reaction of the invention may also be conducted in the presence of a tetraalkylammonium fluoride-hydrogen fluoride such as tetrabutylammonium fluoride-hydrogen fluoride. e.g., tetrabutylammonium dihydrogentrifluoride, (C₄H₉)₄NH₂F₃.

In another embodiment, a method for preparing a trifluoromethyl-containing compound, RCF₃, in accordance with the present invention, comprises reacting a carbon-containing compound, represented by R—C(=A)-R^{a}, with an arylsulfur trifluoride, represented by ArSF₃, under conditions where hydrogen fluoride resulting from the reaction itself remain substantially in the reaction mixture, i.e., steps are taken to ensure that HF remains in the reaction (see below).

R and Ar are the same as described previously. A is an oxygen atom, and R^{a} is a hydroxy group. Thus, R—C(=A)-R^{a} is a carboxylic acid represented by RCOOH, and the reaction scheme is described as follows:

RCOOH+ArSF₃→RCF₃

R and Ar are as described above. A huge number of the carboxylic acids exist naturally (and are commercially available) or can be prepared by well-known conventional methods. As mentioned above, ArSF₃ used for the reaction is readily prepared at relatively low cost.

The reaction equation (Eq. 1) and reaction mechanism (Scheme 1) of a carboxylic acid, represented by RCOOH, with arylsulfur trifluoride, represented by ArSF₃, giving a trifluoromethyl-containing compound, are shown in the following:

RCOOH+2ArSF₃→RCF₃+HF+2ArSOF (Eq. 1)

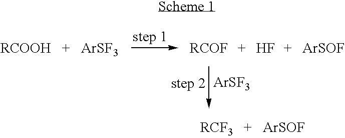

As shown in Scheme 1, the reaction consists of two steps (steps 1 and 2 herein); note that in step 1, hydrogen fluoride (HF) is formed.

This embodiment of the invention is carried out under conditions where some amount of hydrogen fluoride resulting from the reaction of step 1 remains, or is maintained, in the reaction mixture. To get enhanced yields of the trifluoromethyl-containing compounds, at least 10% of hydrogen fluoride generating from step 1 remains or is maintained in the reaction mixture. In some cases 20%, 30%, 40%, 50%, 60%, 70%, 80%, 90%, 95% of the starting amount of hydrogen fluoride remains or is maintained in the reaction mixture. Because the boiling point of hydrogen fluoride is 19.5° C., in order to keep hydrogen fluoride in the reaction (when the reaction is conducted at more than about 19.5° C.), embodiments of this invention can be conducted in a sealed or closed reactor or autoclave, or under pressure so that the hydrogen fluoride is not released from the reaction mixture. The reaction can also be conducted with an effective condenser. This is an unexpected optimization of the reaction embodiments herein.

For optimal product yields, embodiments herein can be performed with the reaction conducted in a sealed or closed reactor or autoclave. However, since the reaction of step 1 can readily occur at or below the boiling point of hydrogen fluoride (bp 19.5° C.), a sealed or closed reactor is not necessarily required. In such cases the reaction is conducted at or below the boiling point of hydrogen fluoride (bp 19.5° C.). However, in order to maintain the hydrogen fluoride resulting from the step 1 in the reaction mixture, a sealed or closed reactor or autoclave may be effective for the reaction of step 2 when conducted at or above the temperature of the boiling point (19.5° C.) of hydrogen fluoride (See Scheme 1).

Since hydrogen fluoride (HF) can react with materials such as glassware, suitable materials for the reactor or autoclave should be utilized, for example, polymers such as fluoro polymers or other HF-resisting polymers, and so on; HF-resisting metals or alloys such as steel, brass, cupper, aluminum, stainless steel, Hastelloy, Monel, and so on can also be used; or HF-resisting polymer-coated glassware, metals or alloys, wherein the polymer neither react nor dissolve with the reaction mixture containing hydrogen fluoride.

The reaction is preferably conducted without a solvent. However, in some cases, a solvent may be used. Suitable solvents for use herein include: hydrocarbons such as hexane, cyclohexane, heptane, octane, nonane, decane, and so on; halocarbons such as methylene chloride, chloroform, carbon tetrachloride, dichloroethane, trichloroethane, tetrachloroethane, perfluorohexane, perfluoroheptane, perfluorooctane, perfluorononane, perfluoro(methylcyclohexane), perfluoro-1-methyldecaline, perfluoro-2-butyltetrahydrofuran, Fluorinart® FC-40˜FC-104, and so on; aromatics such as nitrobenzene, hexafluorobenzene, benzotrifluoride, bis(trifluoromethyl)benzene, and so on; or mixtures of two or more of the above solvents.

As shown in Scheme 1 above, the reaction of the invention consists of two steps referred to as, steps 1 and 2. In order to conduct the reactions safely and obtain good product yields, the reaction temperature for step 1 can be chosen in the range of from about −80° C. to about +40° C. and the reaction temperature for step 2 can be chosen in the range of from about +40° C. to about +200° C. More preferably, the reaction temperature for step 1 is from about −30° C. to about room temperature, and that for step 2 is about +50° C. to from about +150° C. As such, since the reaction of step 1 can be fast, the reaction of step 1 can at least partially occur when the carboxylic acid and ArSF₃ are mixed at the temperature as mentioned above, and hence, after the mixing, the reaction mixture can be heated to the temperature needed for step 2.

In order to obtain good product yield, the amount of ArSF₃ is about 2 mole or more per mole of RCOOH. Preferably, about 2 to about 5 moles of ArSF₃ can be used, and more preferably about 2 to about 3.5 moles can be used, especially where cost is a concern.

Reaction time varies dependent upon reaction temperature, and the types and amounts of substrate, reagent, and solvent present. As such, reaction time is generally determined as the amount of time required to complete a particular reaction, but the total reaction time of steps 1 and 2 can be from about 0.1 hours to about several days.

In alternative embodiments, the present methods include preparation of compounds having two or more trifluoromethyl groups from compounds having two or more carboxyl groups represented by R(COOH)ₙ.

For example, Scheme 2 shows reaction of isophthalic acid (i) with phenylsulfur trifluoride (PhSF₃) according to the present invention.

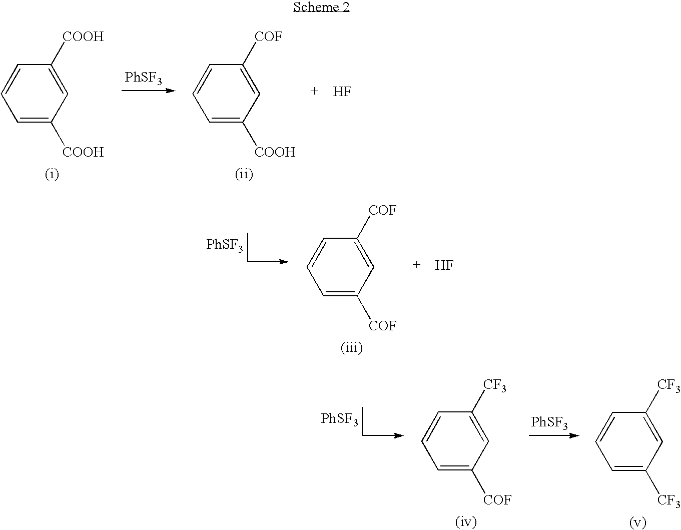

The reaction shown in Scheme 2 proceeds stepwise; compounds (i)→(ii)→(iii)→(iv)→(v). Therefore, compound (iv) is considered as a product from a starting material (i) or (ii) and compound (v) is also considered to be a product from (i) or (ii) as a starting material. Preparation of (iv) and (v) are included in the methods of the present invention. Example 17 (see below) shows the production of compound (v) from a starting material, isophthalic acid (i).

In the case of using R(COOH)ₙ where all the COOH groups are converted to CF₃ groups, the amount of ArSF₃ used is about 2 n moles or more per mole of R(COOH)ₙ. Preferably, 2 n to 5 n moles of ArSF₃ can be used, and more preferably, 2 n to about 3.5 n moles can be used, especially where cost is a concern.

In another embodiment herein, preparation of trifluoromethyl-containing compound, RCF₃, comprises reacting a carbonyl-containing compound, represented by R—C(=A)-R^{c}, with an arylsulfur trifluoride, represented by ArSF₃, in the presence of a mixture of hydrogen fluoride and an amine compound(s).

R and Ar are the same as above. A is an oxygen atom, and R^{c} is a hydroxyl group or a halogen atom. Thus, in this case, R—C(=A)-R^{c} is a carbonyl-containing compound, represented by R—C(═O)—R^{c}, and the reaction scheme is described in the followings:

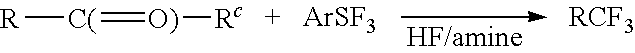

R and Ar are the same as above. A halogen atom for R^{c} can be a fluorine atom, chlorine atom, bromine atom, or iodine atom. Fluorine and chlorine atoms are preferable. A large number of carboxylic acids, represented by R—C(═O)—R^{c}, wherein R^{c}═OH, exist in nature and are commercially available, or can be prepared by well-known conventional methods. Carbonyl halides, when R^{c} is a halogen atom, are commercially available or can be derived from carboxylic acids or other compounds by well-known conventional methods. As mentioned above, ArSF₃ used for the reaction can easily be prepared at relative low cost.

As a source of R—C(═O)—R^{c}, acid anhydrides represented by R—C(═O)—O—C(═O)—R, can be used as acid anhydrides can react with a mixture of hydrogen fluoride and an amine compound(s) to form R—C(═O)—R^{c}, (R^{c}═OH), and R—C(═O)—R^{c} (R^{c}═F), as shown in the following reaction equation [see, J. Org. Chem., Vol. 44, 3872-3881 (1979), incorporated by reference herein]:

R—C(O)—O—C(═O)—R+(HF)ₘ/amine→RCOOH+RCOF+(HF)ₘ₋₁/amine.

Therefore, embodiments herein include usage of acid anhydrides represented by R—C(═O)—O(C═O)—R in the reactions.

Preferable amine compound(s) for use herein include pyridines such as pyridine, each isomer (α, β, or γ-isomer) of methylpyridine, each isomer of dimethylpyridine, each isomer of trimethylpyridine, each isomer of chloropyridine, and so on; alkylamines such as trimethylamine, triethylamine, tripropylamine, tributylamine, and so on; or a mixture of two or more amine compounds as mentioned above.

Preferable examples of a mixture of hydrogen fluoride and amine compound(s), are exemplified as a mixture of hydrogen fluoride and pyridine, a mixture of hydrogen fluoride and each isomer or mixture of methylpyridine, a mixture of hydrogen fluoride and each isomer or mixture of dimethylpyridine, a mixture of hydrogen fluoride and each isomer or mixture of trimethylpyridine, a mixture of hydrogen fluoride and trimethylamine, a mixture of hydrogen fluoride and triethylamine, a mixture of hydrogen fluoride and tripropylamine, a mixture of hydrogen fluoride and tributylamine, and so on. Among them, a mixture of hydrogen fluoride and pyridine is most preferable when availability and product yield are considered.

Mixtures of hydrogen fluoride and amine compound(s) are safer and easier to handle than hydrogen fluoride alone, (which is toxic), because the mixture has a boiling point (or temperature at which hydrogen fluoride evaporates) higher than the boiling point (19.5° C.) of hydrogen fluoride alone. A toxic compound (HF in this case) whose boiling point roughly room temperature has a serious problem in safety of handling. As such, the higher the boiling point, the safer and easier a reaction is conducted. The boiling point of a mixture of hydrogen fluoride and amine compound(s) is dependent on the ratio of each constituent. The smaller the amount of the amine compound in the mixture, the closer the boiling point is to 19.5° C. (hydrogen fluoride's boiling point). It is preferable that the molar ratio of hydrogen fluoride/amine compound(s) be 22:1 or less from the standpoint of handling. It is preferable that the ratio be 3:1 or more from the standpoint of the product yield. Therefore, the molar ratio of hydrogen fluoride/amine compound(s) is preferably selected in the range of from about 3:1 to about 22:1, and more preferably, from about 5:1 to about 16:1. Furthermore, a molar ratio of about 5:1 to about 16:1 mixture of hydrogen fluoride:pyridine is preferable, an about 7:1 to about 12:1 mixture of hydrogen fluoride and pyridine is more preferable, and an about 9:1 (about 70 wt %:30 wt %) mixture of hydrogen fluoride and pyridine is most preferable because of availability and high product yields.

The reaction(s) above can preferably be conducted without a solvent. However, in some cases, a solvent is used. Preferable solvents include hydrocarbons such as hexane, cyclohexane, heptane, octane, nonane, decane, and so on; halocarbons such as methylene chloride, chloroform, carbon tetrachloride, dichloroethane, trichloroethane, tetrachloroethane, perfluorohexane, perfluoroheptane, perfluorooctane, perfluorononane, perfluoro(methylcyclohexane), perfluoro-1-methyldecaline, perfluoro-2-butyltetrahydrofuran, Fluorinart® FC-40˜FC-104, and so on; and aromatics such as nitrobenzene, hexafluorobenzene, benzotrifluoride, bis(trifluoromethyl)benzene, and so on: mixtures of two or more of the above mentioned solvents can be combined for use as well.

In order to obtain optimal product yield, ArSF₃ is used in an amount of about 1 mole or more per mole of R—C(═O)—R^{c}, wherein R^{c}=a halogen atom. Preferably, about from 1 to about 5 moles of ArSF₃ can be used per mole of R—C(═O)—R^{c}, and more preferably about 1 to about 3.5 moles ArSF₃ per mole R—C(═O)—R^{c} can be used, especially where cost is a concern. Alternatively, for one mole of R—C(═)—OH, the amount of ArSF₃ is about 2 mole or more. Preferably, about 2 to about 5 moles of ArSF₃ can be used per mole of R—C(═O)—OH, and more preferably about 2 to about 3.5 moles ArSF₃ per mole of R—C(═O)—OH can be used, especially where cost is of concern.

A catalytic to large excess of a mixture of hydrogen fluoride and amine compound(s) can be used for the above reaction. In order to obtain good product yield, with shorter reaction time, the preferable amount of mixture is to include about 0.2 to about 50 moles of hydrogen fluoride for every mole of ArSF₃. More preferably, the amount is about 0.5 to about 25 moles of hydrogen fluoride per mole of ArSF₃, and furthermore preferably about 0.5 to about 10 moles hydrogen fluoride per mole of ArSF₃, especially where cost is a relative concern.

In the case of R[—C(═O)—R^{c}]ₙ (R^{c}=a halogen atom) where all the C(═O)—R^{c} groups are converted to CF₃ groups, the amount of ArSF₃ used in the reaction is about in moles or more for every mole of R[—C(═O)—R^{c}]ₙ. Preferably, about 1 n to about 5 n moles of ArSF₃ can be used under these conditions, and more preferably, about in to about 3.5 n moles can be used under these conditions, especially where cost is of concern. In the case of R[—C(═O)—R^{c}]ₙ (R^{c}=a hydroxy group) and that all the C(═O)—R^{c} groups are converted to CF₃ groups, the amount of ArSF₃ used is about 2 n moles or more for one mole of R[—C(═O)—R^{c}]ₙ. Preferably, about 2 n to about 5 n moles of ArSF₃ can be used, and more preferably, about 2 n to about 3.5 n moles can be used, especially where cost is a concern.

The reaction can be conducted in an open reactor or in a sealed (closed) reactor.

In the case of R—C(═O)—R^{c}, wherein R^{c}═OH, the reaction of the invention consists of two reactions, steps 1 and 2 as shown in Scheme 1 (above). In order to conduct the two reactions safely and obtain good product yields, the reaction temperature for step 1 can be chosen in the range of from about −80° C. to about +40° C., and the reaction temperature for step 2 can be chosen in the range of from about room temperature to about +200° C. More preferably, the reaction temperature for step 1 is from about −30° C. to about room temperature, and for step 2 is about from room temperature to about +150° C., furthermore preferably for step 2, from about +40° C. to about +100° C. Since the reaction of step 1 can be relatively fast, the reaction of step 1 can at least partially occur when the carboxylic acid and ArSF₃ are mixed at the temperature as mentioned above, and hence, after the mixing, the reaction mixture can be heated to the temperature needed for the step 2.

A mixture of hydrogen fluoride and an amine compound(s) significantly affect the reaction of step 2 in a positive way, but the mixture is not necessarily needed for step 1, due to its relative speed. Therefore, a mixture of hydrogen fluoride and an amine compound(s) may be added to the reaction mixture after RCOOH reacts or mixes with ArSF₃.

In order to obtain optimal product yield for R—C(═O)—R^{c}, wherein R^{c}=a halogen atoms, the reaction temperature is selected in the range of from about 0° C. to about +200° C. More preferably, the reaction temperature can be selected in the range of from about room temperature to about +150° C., furthermore preferably, from about room temperature to about +100° C.

For embodiments using an open reactor, it is preferable that the reaction temperature be maintained below the temperature at which hydrogen fluoride in the mixture boils or significantly evaporates. However, a sealed or closed reactor is preferable when the reaction temperature is close to or higher than the temperature at which hydrogen fluoride in the mixture boils or evaporates. As such, the type of reactor, open or sealed, is directly associated with the reaction temperature.

The reaction time varies dependent upon reaction temperature, the types of reactors, and the types and amounts of substrate, reagent, and solvent present. As such, reaction time is generally determined as the amount of time required to complete a particular reaction, but can be from about 0.1 hours to about several days.

Methods of the invention are simple, unexpectedly safe and easily applicable to industrial production solutions as compared to conventional methodologies. Carbon-containing compounds, represented by R—C(=A)-R^{a}, as starting materials are easily commercially available or prepared via known techniques in the art. Arylsulfur trifluorides used in the present invention can be easily prepared in high yields from inexpensive diphenyl disulfide or primary alkyl-substituted diphenyl disulfides with cheaper reagents, potassium fluoride and chlorine gas, according to the known methods mentioned previously. In addition, arylsulfur trifluorides herein show very high thermal stability as compared to the conventional SF₃ reagent such as diethylaminosulfur trifluoride (Et₂NSF₃; DAST) and bis(2-methoxyethyl)aminosulfur trifluoride [(CH₃OCH₂CH₂)₂NSF₃; Deoxo-Fluor®]. This enhanced stability provides significant benefits over those conventional reagents.

Table 2 provides thermal analysis data for PhSF₃ and p-CH₃C₆H₄SF₃ as used in accordance with the present invention, together with DAST and Deoxo-Fluor® (conventional methodology). Decomposition temperature and exothermic heat (−ΔH) of each compound was determined using Differential Scanning Spectroscopy, i.e., using a Differential Scanning Spectrometer (DSC). The decomposition temperature is the temperature at which onset of decomposition begins, and the exothermic heat is the amount of heat that results from the compounds decomposition. In general, a higher decomposition temperature and lower exothermic heat value provide compounds having greater thermal stability and provide greater safety.

Table 2 illustrates that compounds of the present invention, phenylsulfur trifluoride and p-methylphenylsulfur trifluoride, show very high decomposition temperature and low exothermic heat values as compared to conventional fluorinating agents, DAST and Deoxo-Fluor®. This data illustrates that embodiments of the present invention have greatly improved and unexpected safety over other useful conventional methods, e.g., DAST and Deoxo-Fluor®.

TABLE 2
Thermal Analysis Data of Phenylsulfur Trifluoride (PhSF₃),
p-CH₃C₆H₄SF₃, DAST, and Deoxo-Fluor ®

| | Decomposition | |
|---|---|---|
| Compound | temp. (° C.) | −ΔH(J/g) |
| PhSF₃ | 305 | 826 |
| p-CH₃C₆H₄SF₃ | 274 | 1096 |
| (C₂H₅)₂NSF₃ (DAST) | ~140 | 1700 |
| (CH₃OCH₂CH₂)₂NSF₃ (Deoxo-Fluor ®) | ~140 | 1100 |

According to the present invention, the trifluoromethyl-containing compounds can be safely, easily, selectively and cost-effectively produced from available starting materials.

The following examples will illustrate the present invention in more details, but it should be understood that the present invention is not deemed to be limited thereto.

### EXAMPLES

### Example 1

### Production of Difluoromethylene-Containing Compounds

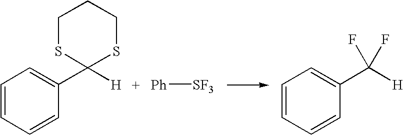

The reaction of Example 1 was performed in dry atmosphere under nitrogen. A solution of 2-phenyl-1,3-dithiane (85 mg, 0.47 mmol) in 1 mL of dry methylene chloride was dropwise added to a solution of phenylsulfur trifluoride (200 mg, 1.2 mmol) in 1 mL of dry methylene chloride. The reaction was performed in a fluoropolymer (PFA) reactor. The reaction mixture was stirred at room temperature for 2 hours. The reaction mixture was analyzed by ¹⁹F-NMR, showing that (difluoromethyl)benzene was produced in 99% yield. The product was identified by comparison with an authentic sample. ¹⁹F NMR (CDCl₃ as a solvent; CFCl₃ as a standard) for PhCF₂H: −110.5 ppm (d, J=56 Hz, CF₂).

### Examples 2-8

### Production of Difluoromethylene-Containing Compounds

Examples 2-8 were conducted under conditions as shown in Table 3 in a similar manner as for Example 1. The results are shown in Table 3 together with Example 1. The products were identified by spectral analyses and/or by comparison with authentic samples. ¹⁹F NMR data (ppm; CDCl₃ as a solvent; CFCl₃ as a standard) of the products are shown in Table 3.

TABLE 3
Preparation of Various Difluoromethylene-containing Compounds with ArSF₃
and R¹—C(R³)(R⁴)—R².

| | | | | | | Product, | | ¹⁹F |
|---|---|---|---|---|---|---|---|---|
| | ArSF₃ | R¹—C(R³)(R⁴)—R² | Solvent | Temp | Time | R¹CF₂R² | Yield* | NMR |
| Ex. 1 | PhSF₃ (1.2 mmol) | 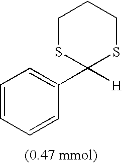 | CH₂Cl₂ (1 mL) | r.t. | 2 h | PhCF₂H | 94% | −110.5 (d, J = 56 Hz) |
| Ex. 2 | p- CH₃C₆H₄SF₃ (1.3 mmol) | 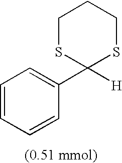 | CH₂Cl₂ (1 mL) | r.t. | 2 h | PhCF₂H | 95% | −110.5 (d, J = 56 Hz) |
| Ex. 3 | PhSF₃ | PhC(═S)Ph | CH₂Cl₂ | r.t. | 2 h | PhCF₂Ph | quant | −88.7 (s) |
| | (4.10 mmol) | (1.64 mmol) | (1 mL) | | | | | |
| Ex. 4 | PhSF₃ | PhC(═S)OCH₃ | CH₂Cl₂ | r.t. | 3 h | PhCF₂OCH₃ | 98% | −72.2 (s) |
| | (3.45 mmol) | (1.39 mmol) | (1 mL) | | | | | |
| Ex. 5 | PhSF₃ | n- | CH₂Cl₂ | r.t. | 20 h | n- | 80% | −77.8 (s) |
| | (2.55 mmol) | C₇H₁₅C(═S)OCH₃ | (3 mL) | | | C₇H₁₅CF₂OCH₃ | | |
| | | (1.66 mmol) | | | | | | |
| Ex. 6 | PhSF₃ (2.85 mmol) | 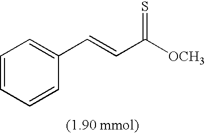 | CH₂Cl₂ (4 mL) | r.t. | 20 h | 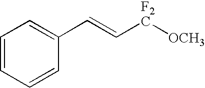 | 91% | −69.6 (s) |
| Ex. 7 | PhSF₃ (2.35 mmol) | 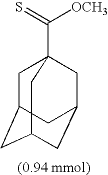 | CH₂Cl₂ (3 mL) | r.t. | 4 h | 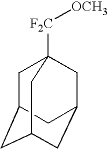 | quant | −94.6 (s) |
| Ex. 8 | PhSF₃ | PhC(═S)SCH₃ | CH₂Cl₂ | r.t. | 5 h | PhCF₂SCH₃ | 75% | −75.1 (s) |
| | (3.62 mmol) | (0.72 mmol) | (1 mL) | | | | | |

*quant = a quantitative yield.

The products, difluoromethylene-containing compounds, can easily be separated from arylsulfur compounds, formed from ArSF₃, by washing with an aqueous solution, such as aqueous sodium carbonate solution, since the arylsulfur compounds are soluble in the aqueous solution. Excess ArSF₃ left in the reactions can also be easily separated from the difluoromethylene-containing compounds by washing with the aqueous solution. Thus, embodiments of the invention have a great advantage in the separation process after the reaction.

As shown from Examples 1-8 in Table 3, it has been unexpectedly shown that phenylsulfur trifluoride or a one-primary alkyl-substituted phenylsulfur trifluoride fluorinates the sulfur-containing compounds represented by R¹—C(R³)(R⁴)—R² to provide a high yield of difluoromethylene-containing compounds. Reactivity of phenylsulfur trifluoride has been shown to be low [see J. Am. Chem. Soc., Vol. 84, pp. 3058-3063 (1962)]. As mentioned above, phenylsulfur trifluoride and one-primary alkyl-substituted phenylsulfur trifluorides have high thermal stability and can be produced at low cost, and the sulfur-containing compounds are easily available. These high safety, low cost, simple procedure, and high yields of product embodiments are particularly significant for industrial application.

### Example 9

### Production of Trifluoromethyl-Containing Compounds

PhC(═S)SCH₃+PhSF₃→PhCF₃

This reaction was performed in anhydrous atmosphere under nitrogen. Phenylsulfur trifluoride (264 mg, 1.59 mmol) and methyl dithiobenzoate (53.5 mg, 0.31 mmol) were put in a fluoropolymer (PFA) tube (reactor) at room temperature, and then the tube was sealed. The reaction mixture was heated at 70° C. for 22 hours. The reaction was then cooled to room temperature and analyzed by ¹⁹F-NMR. The analysis showed that benzotrifluoride was produced at 85% yield. The product was identified by comparison with an authentic sample. ¹⁹F NMR for PhCF₃ (CDCl₃); −62.6 ppm (s, CF₃).

### Examples 10-12

### Production of Trifluoromethyl-Containing Compounds

Reactions for Examples 10-12 were performed in a similar manner to Example 9 under reaction conditions as shown in Table 4. In Examples 10 and 11, a sealed reactor was used. In Example 12, an open reactor was used. The results are shown in Table 4 together with Example 9. The products were identified by comparison with authentic samples or spectral analyses. In Example 10, ¹⁹F NMR for PhOCF₃ (CDCl₃); −57.8 ppm (s, CF₃). In Example 11, ¹⁹F NMR for n-C₁₀H₂₁OCF₃ (CDCl₃); −60.5 ppm (s, CF₃). In Example 12, ¹⁹F NMR for 2-pyridyl-N(CH₃)CF₃ (CDCl₃); −57.9 ppm (s, CF₃).

TABLE 4
Preparation of Various Trifluoromethyl-containing Compounds with ArSF₃ and
Thiocarbonyl-containing Compounds, R—C(═S)—SR^{b}

| | | | | | | | Product, | |
|---|---|---|---|---|---|---|---|---|
| | ArSF₃ | R—C(═S)—SR^{b} | Reactor | Solvent | Temp | Time | RCF₃ | Yield |
| Ex. 9 | PhSF₃ | PhC(═S)SCH₃ | Sealed | Non | 70° C. | 22 h | PhCF₃ | 85% |
| | (1.59 mmol) | (0.31 mmol) | reactor | | | | | |
| Ex. | p-CH₃C₆H₄SF₃ | PhOC(═S)SCH₃ | Sealed | Non | 60° C. | 15 h | PhOCF₃ | 77% |
| 10 | (1.27 mmol) | (0.42 mmol) | reactor | | | | | |
| Ex. | PhSF₃ | n-C₁₀H₂₁OC(═S)SCH₃ | Sealed | Non | 70° C. | 22 h | n-C₁₀H₂₁OCF₃ | 67% |
| 11 | (1.66 mmol) | (0.33 mmol) | reactor | | | | | |
| Ex. 12 | PhSF₃ (3.16 mmol) | 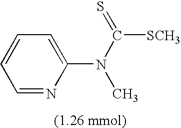 | Open reactor | Non | r.t.¹⁾ | 24 h | 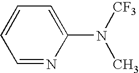 | 98% |

¹⁾r.t. = room temperature.

### Example 13

### Production of Trifluoromethyl-Containing Compounds

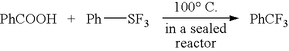

The reaction was performed in anhydrous atmosphere under nitrogen. Benzoic acid (0.34 mmol) was added portion by portion to phenylsulfur trifluoride (0.848 mmol) in a fluoropolymer (PFA) tube (reactor) at room temperature. When the two reactants were mixed, a mild exothermic reaction occurred. After the addition, the tube was sealed. The reaction mixture was heated for 2 hours at 100° C. After 2 hours, the reaction mixture was cooled to room temperature and analyzed by ¹⁹F-NMR. The analysis showed that benzotrifluoride was produced in 90% yield. The product was identified by comparison with an authentic sample. ¹⁹F NMR for PhCF₃ (CDCl₃); −62.6 ppm (s, CF₃).

### Examples 14-17 and Comparative Examples 18-21

### Production of Trifluoromethyl-Containing Compounds

Examples 14-17 were conducted in a similar manner to Example 13 under the reaction conditions as shown in Table 5. The reaction temperatures shown in Table 5 are the temperature at which the reaction mixture was heated after the two reactants were mixed at room temperature. The reaction times shown in Table 5 are the times for which the reaction mixture was heated at the reaction temperature shown. The results are shown in Table 5 together with Example 13. The products were identified by comparison with authentic samples or spectral analyses. In Example 14, ¹⁹F NMR for n-C₁₀H₂₁CF₃ (CDCl₃); −66.4 ppm (s, CF₃). In Example 15 and Comparative Examples 18-20, ¹⁹F NMR for PhCF₃ (CDCl₃); −62.6 ppm (s, CF₃). In Example 16, ¹⁹F NMR for p-(n-C₇H₁₅)C₆H₄CF₃ (CDCl₃); −62.1 ppm (s, CF₃). In Example 17, ¹⁹F NMR for 1,3-diCF₃C₆H₄ (CDCl₃); −62.9 ppm (s, CF₃).

Comparative Examples 18 and 19 were conducted in a similar manner to Example 13 except that the reaction was carried out in an open reactor. In an open reaction, hydrogen fluoride formed during the reaction completely, or almost completely, escaped from the reaction mixture (heated at 100° C. since hydrogen fluoride's boiling point is 19.5° C.). Comparative Examples 20 and 21 were conducted in a similar manner to Example 13. The results of Comparative Examples 18-21 are shown in Table 5.

TABLE 5
Preparation of Various Trifluoromethyl-containing Compounds with ArSF₃ and
Carboxylic Acids, and the Results of Comparative Examples 18-21

| | ASF₃ | RCOOH | Reactor | Temp. | Time | Product, RCF₃ | Yield |
|---|---|---|---|---|---|---|---|
| Ex. 13 | PhSF₃ | PhCOOH | Sealed | 100° C. | 2 h | PhCF₃ | 90% |
| | (2.7 mmol) | (1.08 mmol) | reactor | | | | |
| Ex. 14 | PhSF₃ | n-C₁₁H₂₃COOH | Sealed | 100° C. | 2 h | n-C₁₁H₂₃CF₃ | 83% |
| | (1.92 mmol) | (0.77 mmol) | reactor | | | | |
| Ex. 15 | p- | PhCOOH | Sealed | 100° C. | 2 h | PhCF₃ | 67% |
| | CH₃C₆H₄SF₃ | (0.46 mmol) | reactor | | | | |
| | (1.16 mmol) | | | | | | |
| Ex. 16 | PhSF₃ | p-(n- | Sealed | 100° C. | 4 h | p-(n-C₇H₁₅)C₆H₄CF₃ | 96% |
| | (4.03 mmol) | C₇H₁₅)C₆H₄COOH | reactor | | | | |
| | | (1.36 mmol) | | | | | |
| Ex. 17 | PhSF₃ | Isophthalic acid | Sealed | 100° C. | 2 h | 1,3-bis(trifluoromethyl)- | 93% |
| | (3.19 mmol) | (0.70 mmol) | reactor | | | benzene | |
| Comp. | PhSF₃ | PhCOOH | Open | 100° C. | 2 h | PhCF₃ | 28% |
| Ex. 18 | (1.8 mmol) | (0.72 mmol) | reactor | | | | |
| Comp. | PhSF₃ | PhCOOH | Open | 100° C. | 24 h | PhCF₃ | 49% |
| Ex. 19 | (6.4 mmol) | (2.1 mmol) | reactor | | | | |
| Comp. | PhSF₃ | PhCOF | Sealed | 100° C. | 2 h | PhCF₃ | ~1%¹⁾ |
| Ex. 20 | (2.0 mmol) | (0.80 mmol) | reactor | | | | |
| Comp. Ex. 21 | PhSF₃ (1.86 mmol) | 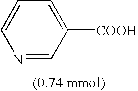 | Sealed reactor | 100° C. | 2 h | 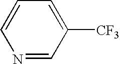 | 0%²⁾ |

¹⁾95% of PhCOF (a starting material) remained intact.
²⁾¹⁹F NMR analysis showed that 3-pyridylcarbonyl fluoride was formed in 45% yield.

As shown from Examples 9-17 in Tables 4 and 5, it has been unexpectedly shown that the present invention's method with phenylsulfur trifluoride or a one-primary alkyl-substituted phenylsulfur trifluoride provides a strikingly high yield of trifluoromethyl-containing compounds compared to the report that, when an alkylcarboxylic acid was reacted with phenylsulfur trifluoride at 110-125° C. for 2 hours at atmospheric pressure (open reactor), a (trifluoromethyl)alkane was produced at only 28% yield [see J. Am. Chem. Soc., Vol. 84, pp. 3058-3063 (1962)].

Furthermore, this method is unexpectedly conducted at lower cost and with higher productiveness than the recently published method with multi-substituted phenylsulfur trifluorides, which are activated by two or more alkyl substituents (U.S. Pat. No. 7,265,247 B1).

The present invention's arylsulfur trifluorides, phenylsulfur trifluoride and one-primary alkyl-substituted phenylsulfur trifluorides, which are not activated by two or more multi-alkyl substituents, are cheaper and have less molecular weight than the multi-substituted phenylsulfur trifluorides. The smaller the molecular weight, the bigger the productivity per weight of the reagent.

Comparison of Examples 13-17 and Comparative Examples 18 and 19 demonstrate that the present invention provide an unexpectedly improved method. A method (sealed reactor; Example 13) of this invention gave 90% yield of the product after 2 hours at 100° C., in contrast, the open reactor (Camp. Ex. 18 and 19) afforded only 28% yield after 2 hours, and 49% even after 24 hours at the same temperature. Comparative Example 20 shows that actually no benzoyl fluoride was converted to benzotrifluoride under the same condition as Example 13, demonstrating that hydrogen fluoride formed by step 1 of this invention's reaction (Scheme 1) is crucial for the reaction of the invention. Comparative Example 21 shows that pyridine-3-carboxylic acid is not converted to 3-(trifluoromethyl)pyridine by the reaction conditions of the invention, providing another proof that the free hydrogen fluoride is crucial for the reaction of the invention, because the hydrogen fluoride generating according to step 1 is deactivated by a basic nitrogen site of pyridine-3-carboxylic acid, forming 1 as shown in Scheme 3.

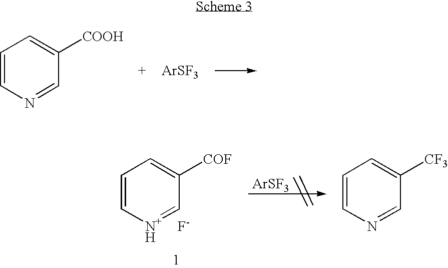

Under these reaction conditions, 3-pyridyl group is an organic moiety which may hurt the reaction of the invention. However, 3-pyridyl group can be converted to a non-harmful group by adding a thoroughly strong Lewis acid or Brönsted acid or by any other chemical transformation.

### Example 22

### Production of Trifluoromethyl-Containing Compounds

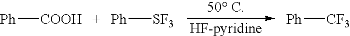

The reaction shown in Example 22 was performed in anhydrous atmosphere under nitrogen. At room temperature, benzoic acid (212 mg, 1.73 mmol) and phenylsulfur trifluoride (865 mg, 5.21 mmol) were mixed portion by portion in a fluoropolymer (PFA) reactor with a condenser, a nitrogen gas inlet connected to a nitrogen cylinder, and a nitrogen gas outlet connecting to air atmosphere. When the two reactants were mixed, a mild exothermic reaction occurred. After mixing, 1.2 mL of an about 70%:30% (wt/wt) mixture of hydrogen fluoride and pyridine (from Sigma-Aldrich) were added to the mixture. The reaction mixture was then heated at 50° C. for 24 hours under nitrogen atmosphere at atmospheric pressure (open reactor). After 24 hours, the reaction mixture was cooled to room temperature and ¹⁹F-NMR analysis of the reaction mixture was performed, indicating that benzotrifluoride was obtained in 95% yield. The product was identified by comparison with an authentic sample. ¹⁹F NMR for PhCF₃ (CDCl₃); −62.6 ppm (s, CF₃).

¹⁹NMR analysis clearly showed that the first product of this reaction is benzoyl fluoride (PhCOF), which is then converted to the final product, benzotrifluoride. Therefore, this experiment (Example 22) is an example of the reaction of the conversion of PhCOF to PhCF₃, shown in the following:

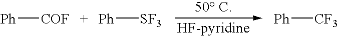

### Examples 23-25

### Production of Trifluoromethyl-Containing Compounds

Examples 23-25 were conducted in a similar manner to Example 22 under the reaction conditions as shown in Table 6. The reaction temperatures shown in Table 6 are the temperatures at which the reaction mixture was heated after the two reactants were mixed at room temperature. Table 6 shows the results of Examples 23-25 together with Example 22. The products were identified by comparison with authentic samples or spectral analyses. In Example 23, ¹⁹F NMR for PhCF₃ (CDCl₃); −62.6 ppm (s, CF₃). In Example 24, ¹⁹F NMR for p-(n-C₇H₁₅)C₆H₄CF₃ (CDCl₃); −62.1 ppm (s, CF₃). In Example 25, ¹⁹F NMR for n-C₁₀H₂₁CF₃ (CDCl₃); −66.4 ppm (s, CF₃).

TABLE 6
Preparation of Various Trifluoromethyl-containing Compounds with ArSF₃ and Carboxylic
Acids in the Presence of a Mixture of Hydrogen Fluoride and an Amine Compound(s)

| | ASF₃ | RCOOH | HF/amine | Temp | Time | Product, RCF₃ | Yield |
|---|---|---|---|---|---|---|---|
| Ex. 22 | PhSF₃ | PhCOOH | HF/pyridine (about | 50° C. | 24 h | PhCF₃ | 95% |
| | (5.21 mmol) | (1.73 mmol) | 70 wt %/30 wt %) (1.2 mL) | | | | |
| Ex. 23 | PhSF₃ | PhCOCl | HF/pyridine (about | 50° C. | 24 h | PhCF₃ | 93% |
| | (2.76 mmol) | (0.92 mmol) | 70 wt %/30 wt %) (0.5 mL) | | | | |
| Ex. 24 | PhSF₃ | p-(n- | HF/pyridine (about | 50° C. | 24 h | p-(n-C₇H₁₅)C₆H₄CF₃ | 92% |
| | (4.52 mmol) | C₇H₁₅)C₆H₄COOH | 70 wt %/30 wt %) (0.8 mL) | | | | |
| | | (1.51 mmol) | | | | | |
| Ex. 25 | PhSF₃ | n-C₁₁H₂₃COOH | HF/pyridine (about | 50° C. | 24 h | n-C₁₁H₂₃CF₃ | Quant* |
| | (4.12 mmol) | (1.37 mmol) | 70 wt %/30 wt %) (0.7 mL) | | | | |

*quant = a quantitative yield.

It is understood for purposes of this disclosure, that various changes and modifications may be made to the invention that are well within the scope of the invention. Numerous other changes may be made which will readily suggest themselves to those skilled in the art which are encompassed in the spirit of the invention disclosed herein and as defined in the appended claims.

This specification contains numerous citations to references such as patents, patent applications, and publications. Each is hereby incorporated by reference for all purposes.

What is claimed is:

1. A method for preparing a difluoromethylene-containing compound, represented by R¹CF₂R², comprising reacting a sulfur-containing compound, represented by R¹—C(R³)(R⁴)—R², with an arylsulfur trifluoride, represented by ArSF₃;
in which R¹ is an organic moiety; R² is a hydrogen atom or an organic moiety; R³ and R⁴ each is independently an alkylthio group, an arylthio group, or an aralkylthio group, wherein R³ and R⁴ may be combined or connected via an alkylene chain and/or a hetero atom(s); or R³ and R⁴ combine to form a sulfur atom; and Ar is a phenyl group or phenyl group having a primary alkyl substituent, wherein the primary alkyl substituent has from one to eight carbon atoms.

2. The method of claim 1, wherein the primary alkyl substituent has from one to four carbon atoms.

3. The method of claim 1, wherein the arylsulfur trifluoride is selected from a group consisting of phenylsulfur trifluoride, p-methylphenylsulfur trifluoride, p-ethylphenylsulfur trifluoride, p-(n-propyl)phenylsulfur trifluoride, p-(n-butyl)phenylsulfur trifluoride, and p-(2-methylpropyl)phenylsulfur trifluoride.

4. The method of claim 1, wherein the arylsulfur trifluoride is selected from a group consisting of phenylsulfur trifluoride and p-methylphenylsulfur trifluoride.

5. The method of claim 1, wherein the arylsulfur trifluoride is phenylsulfur trifluoride.

6. A method for preparing a trifluoromethyl-containing compound, represented by RCF₃, comprising:
reacting a thiocarbonyl-containing compound, represented by R—C(═S)—SR^{b}, with an arylsulfur trifluoride, represented by ArSF₃;
in which R is an organic moiety; R^{b} is a hydrogen atom, an alkyl group, an aryl group, an aralkyl group, a silyl group, a metal atom, an ammonium moiety, a phosphonium moiety, or S—C(═S)—R, and Ar is a phenyl group or phenyl group having a primary alkyl substituent, wherein the primary alkyl substituent has from one to eight carbon atoms.

7. The method of claim 6, wherein the primary alkyl substituent has from one to four carbon atoms.

8. The method of claim 6, wherein the arylsulfur trifluoride is selected from a group consisting of phenylsulfur trifluoride, p-methylphenylsulfur trifluoride, p-ethylphenylsulfur trifluoride, p-(n-propyl)phenylsulfur trifluoride, p-(n-butyl)phenylsulfur trifluoride, and p-(2-methylpropyl)phenylsulfur trifluoride.

9. The method of claim 6, wherein the arylsulfur trifluoride is selected from a group consisting of phenylsulfur trifluoride and p-methylphenylsulfur trifluoride.

10. The method of claim 6, wherein the arylsulfur trifluoride is phenylsulfur trifluoride.

11. A method for preparing a trifluoromethyl-containing compound, represented by RCF₃, comprising reacting a carboxylic acid, represented by RCOOH, with an arylsulfur trifluoride, represented by ArSF₃, under conditions where hydrogen fluoride resulting from the reaction is kept in the reaction;
in which R is an organic moiety, and Ar is phenyl group or phenyl group having a primary alkyl substituent, wherein the primary alkyl substituent has from one to eight carbon atoms.

12. The method of claim 11, wherein the primary alkyl substituent has from one to four carbon atoms.

13. The method of claim 11, wherein the arylsulfur trifluoride is selected from a group consisting of phenylsulfur trifluoride, p-methylphenylsulfur trifluoride, p-ethylphenylsulfur trifluoride, p-(n-propyl)phenylsulfur trifluoride, p-(n-butyl)phenylsulfur trifluoride, and p-(2-methylpropyl)phenylsulfur trifluoride.

14. The method of claim 11, wherein the arylsulfur trifluoride is selected from a group consisting of phenylsulfur trifluoride and p-methylphenylsulfur trifluoride.

15. The method of claim 11, wherein the arylsulfur trifluoride is phenylsulfur trifluoride.

16. The method of claim 11, wherein the reaction is conducted in the absence of a solvent.

17. The method of claim 11, wherein at least a portion of the reaction is conducted in a substantially sealed (closed) reactor.

18. A method for preparing a trifluoromethyl-containing compound, represented by RCF₃, comprising reacting a carbonyl-containing compound, represented by R—C(═O)—R^{c}, with an arylsulfur trifluoride, represented by ArSF₃, in the presence of a mixture of hydrogen fluoride and an amine compound(s);
in which R is an organic moiety, R^{c} is a hydroxyl group or a halogen atom, and Ar is phenyl group or a phenyl group having a primary alkyl substituent, wherein the primary alkyl substituent has from one to eight carbon atoms.

19. The method of claim 18, wherein the primary alkyl substituent has from one to four carbon atoms.

20. The method of claim 18, wherein the arylsulfur trifluoride is selected from a group consisting of phenylsulfur trifluoride, p-methylphenylsulfur trifluoride, p-ethylphenylsulfur trifluoride, p-(n-propyl)phenylsulfur trifluoride, p-(n-butyl)phenylsulfur trifluoride, and p-(2-methylpropyl)phenylsulfur trifluoride.

21. The method of claim 18, wherein the arylsulfur trifluoride is selected from a group consisting of phenylsulfur trifluoride and p-methylphenylsulfur trifluoride.

22. The method of claim 18, wherein the arylsulfur trifluoride is phenylsulfur trifluoride.

23. The method of claim 18, wherein a molar ratio of hydrogen fluoride/amine compound(s) is 22:1 or less.

24. The method of claim 18, wherein the amine compound(s) is pyridine.

25. The method of claim 18, wherein a molar ratio of hydrogen fluoride and pyridine is in the range of 16:1 to 5:1.

26. The method of claim 18, wherein the reaction is conducted in the absence of a solvent.
